(19) United States
(12) Patent Application Publication
(10) Pub. No.: US 2012/0125346 A1
(43) Pub. Date: May 24, 2012

(54) ENDOTRACHEAL CUFF AND TECHNIQUE FOR USING THE SAME

(75) Inventors: Jessica Clayton, San Jose, CA (US); Ahmad Robert Hadba, Wallingford, CT (US); Megan Prommersberger, Wallingford, CT (US); Joshua Stopek, Wallingford, CT (US); Brian Cuevas, Cumming, GA (US)

(73) Assignee: Nellcor Puritan Bennett LLC, Boulder, CO (US)

(21) Appl. No.: 13/363,962

(22) Filed: Feb. 1, 2012

Related U.S. Application Data
(62) Division of application No. 11/713,557, filed on Mar. 2, 2007.

(51) Int. Cl. A61M 16/04; B05D 7/00

(52) U.S. Cl. 128/207.15; 427/2.1

(57) ABSTRACT

An inflatable balloon cuff with a water-swellable coating may be adapted to seal a patient's trachea when associated with an endotracheal tube. The water-swellable coating may enhance a cuffs mechanical pressure seal. The water-swellable coating may be loosely adhered or not adhered to the cuff in order to allow the coating to flow into and seal any leak paths that may form when the cuff is inflated in a patient's trachea.

### BACKGROUND OF THE INVENTION

1. Field of the Invention

The present invention relates to medical devices, and more particularly, to endotracheal devices, such as endotracheal tubes and cuffs.

2. Description of the Related Art

This section is intended to introduce the reader to various aspects of art that may be related to various aspects of the present invention which are described and/or claimed below. This discussion is believed to be helpful in providing the reader with background information to facilitate a better understanding of the various aspects of the present invention. Accordingly, it should be understood that these statements are to be read in this light, and not as admissions of prior art.

In the course of treating a patient, a tube or other medical device may be used to control the flow of air, food, fluids, or other substances into and/or out of the patient. For example, medical devices such as tracheal tubes may be used to control the flow of one or more substances into or out of a patient. In many instances it is desirable to provide a seal between the outside of the tube or device and the interior of the passage in which the tube or device is inserted. In this way, substances can only flow through the passage via the tube or other medical device, allowing a medical practitioner to maintain control over the type and amount of substances flowing into and out of the patient.

Tracheal tubes may be used to control the flow of air or other gases through a patient's trachea. Such tracheal tubes may include endotracheal (ET) tubes or tracheostomy tubes. To seal these types of tracheal tubes, an inflatable cuff may be associated with these tubes. When inflated, the cuff generally expands into the surrounding trachea to seal the tracheal passage around the open lumen of the tube. A high-quality seal against the tracheal passageway allows a patient ventilator to perform efficiently.

However, to fit a range of trachea anatomies and to provide low intra cuff pressure, physicians may use high pressure, low volume cuffs. Such cuffs may provide the advantage of sealing the trachea at lower pressures that are more comfortable for the patient, but have certain associated disadvantages. In such cuffs, the inflated cuff diameters are typically about one and a half times the diameter of the average trachea. Therefore, when inserted in an average-sized trachea, such a cuff is unable to fully expand and will fold in on itself within the trachea. These folds may serve as leak paths that allow mucosal secretions to flow past the cuff and enter the lung. Because mucosal secretions may harbor microbes, it is desirable to prevent such secretions from entering the lungs.

Certain types of cuffs are manufactured from materials that have a lower tendency to form leak paths. For example, high pressure cuffs are typically made of highly elastic materials that may form a relatively smooth seal against the trachea. However, such cuffs have associated disadvantages. Due to their elastic properties, high pressure cuffs are often inflated to at least twice the intracuff pressure of lower pressure cuffs in order to form a sufficient tracheal seal. Such high pressures may cause patient discomfort. Further, the mechanical pressure of the high pressure cuff against the tracheal walls may also cause temporary damage to cilial structures in the trachea that are associated with airway particle clearance.

Attempts have been made to modify low pressure, high volume cuffs with different types of coatings in order to improve their tracheal sealing properties. For example, permanently adhered swellable coatings with limited viscosity have been detailed that are designed to swell within leak paths to seal them and prevent the passage of secretions into the lungs. However, such adhered coatings may present certain problems. For example, adhesion of certain types of hydrogels or certain swellable polymer coatings to a cuff may weaken its material properties, making it more likely to tear or leak.

### SUMMARY

Certain aspects commensurate in scope with the originally claimed invention are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

A medical device is provided that includes an inflatable balloon cuff including a distal opening and a proximal opening, wherein the distal opening and the proximal opening are suitably sized to accommodate a conduit; and a water-swellable coating non-adherently disposed on the balloon cuff, the water-swellable coating including a polymer formed from at least one acrylated prepolymer.

Also provided is a medical device that includes an inflatable balloon cuff including a distal opening and a proximal opening, wherein the distal opening and the proximal opening are suitably sized to accommodate a conduit; and a composition disposed on the balloon cuff, the composition including a copolymer that includes a first monomer, and a second monomer that is different from the first monomer, wherein the first monomer includes 3-sulfopropyl acrylate potassium salt, sodium acrylatc, N-(tris(hydroxyl methyl)methyl)acrylamide, or 2-acrylamido-2-methyl-1-propane sulfonic acid.

Also provided is a method of manufacturing a medical device that includes: providing an inflatable balloon cuff including a distal opening and a proximal opening, wherein the distal opening and the proximal opening are suitably sized to accommodate a conduit; and non-adherently disposing a water-swellable coating on the balloon cuff, the water-swellable coating including a polymer formed from at least one acrylated prepolymer.

Also provided is a method of manufacturing a medical device that includes: providing an inflatable balloon cuff including a distal opening and a proximal opening, wherein the distal opening and the proximal opening are suitably sized to accommodate a conduit; and disposing a composition on the balloon cuff, the composition including a copolymer that includes a first monomer, and a second monomer that is different from the first monomer, wherein the first monomer comprises 3-sulfopropyl acrylate potassium salt, sodium acrylate, N-(tris(hydroxyl methyl)methyl) crylamide, or 2-acrylamido-2-methyl-1-propane sulfonic acid.

Also provided is a medical device that includes: an inflatable balloon cuff including a distal opening and a proximal opening, wherein the distal opening and the proximal opening are suitably sized to accommodate a conduit; and a water-swellable interpenetrating network disposed on the balloon cuff.

Also provided is a method of manufacturing a medical device that includes: providing an inflatable balloon cuff including a distal opening and a proximal opening, wherein the distal opening and the proximal opening are suitably sized to accommodate a conduit; contacting the balloon cuff with a swelled prepolymer solution comprising at least one monomer or at least one oligomer, at least one cross-linker, and at least one initiator, wherein the at least one monomer or the at least one oligomer are adapted to form a hydrogel when polymerized; polymerizing the swelled prepolymer solution to form an interpenetrating hydrogel network on the balloon cuff.

### BRIEF DESCRIPTION OF THE DRAWINGS

Advantages of the invention may become apparent upon reading the following detailed description and upon reference to the drawings in which:

FIG. 1 illustrates an endotracheal tube with an inflatable balloon cuff with a filtration layer in accordance with aspects of the present technique;

FIG. 2 illustrates the inflatable balloon cuff of the present techniques inserted into a patient's trachea;

FIG. 3 illustrates an exemplary water-swellable layer sealing a cuff wrinkle;

FIG. 4 illustrates an exemplary water-swellable layer that has formed an interpenetrating layer with a cuff;

FIG. 5 illustrates an exemplary polyvinyl chloride inflatable balloon cuff of the present techniques with a hydrogel layer;

FIG. 6 illustrates an exemplary polyvinyl chloride inflatable balloon cuff of the present techniques with a polyvinyl pyrrolidone tie layer and a hydrogel layer; and

FIG. 7 illustrates an exemplary polyvinyl chloride inflatable balloon cuff of the present techniques with a hydromer layer and a hydrogel

### DETAILED DESCRIPTION OF SPECIFIC EMBODIMENTS

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

It is desirable to provide a medical balloon, such as an endotracheal cuff, that may have an improved seal when inserted into a patient's trachea. Inflatable cuffs in accordance with this disclosure include water-swellable polymer compositions. These compositions may be applied to a tissue-contacting surface of an endotracheal cuff. Upon contact with the fluid environment of a patient's trachea, the polymer compositions may swell to seal any gaps between the cuff and the tracheal wall. Further, the polymer compositions may also swell in and around any folds that form in the cuff, which may reduce the ability of secretions to flow through such folds into the lungs.

The water-swellable compositions provided herein may be permanently or non-permanently adhered to the surface of the cuff. In such embodiments in which the water-swellable compositions are permanently adhered to the cuffs, the compositions may have material properties that may generally allow the compositions to adequately swell while also preserving the ability of the cuff material to maintain suitable inflation pressures after the coating has been applied. In other embodiments, the water-swellable compositions provided herein are non-adhesively disposed on the cuffs of the present techniques.

The balloon cuffs provided herein may be used in conjunction with any suitable medical device. In certain embodiments, the cuffs as provided herein may be used in conjunction with a catheter, a stent, a feeding tube, an intravenous tube, an endotracheal tube, a circuit, an airway accessory, a connector, an adapter, a filter, a humidifier, a nebulizer, or a prosthetic.

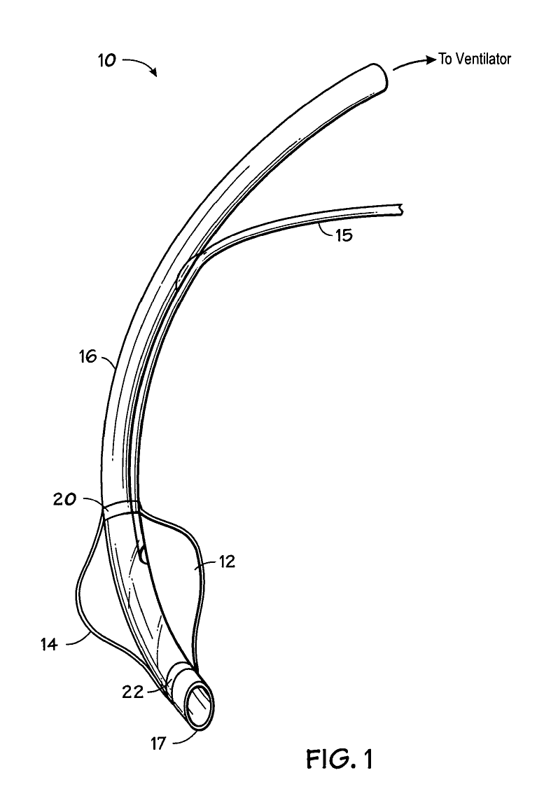

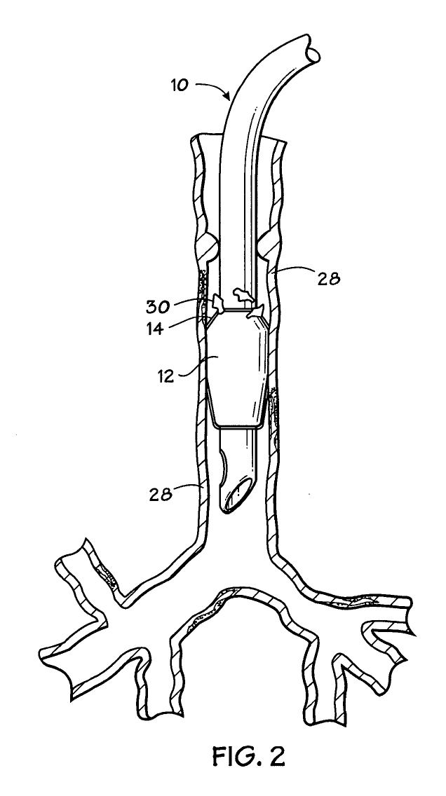

An example of a cuff used in conjunction with a medical device is a cuffed endotracheal tube 10, depicted in FIG. 1. The cuffed endotracheal tube 10 includes an inflatable cuff 12 that may be inflated to form a seal against the trachea wall 28 (see FIG. 2). In certain embodiments, the cuff 12 includes a water-swellable layer 14 that is disposed over the outer surface of the cuff 12. The cuff is disposed on an endotracheal tube 16 that is suitably sized and shaped to be inserted into a patient and allow the passage of air through the endotracheal tube 16. Typically, the cuff is disposed, adhesively or otherwise, towards the distal end 17 of the endotracheal tube 16. The cuff 12 may be inflated and deflated via a lumen 15 in communication with the cuff 12, typically through a hole or notch in the lumen 15. The cuff 12 has a proximal opening 20 and a distal opening 22 formed in the cuff walls sized to accommodate the endotracheal tube 16. The proximal opening 20, located closer to the “machine end” of the tube 16, and a distal opening 22, located closer to the “patient end” of the tube 16, are typically used to mount the cuff 12 to the tube 16.

The cuff 12 may be formed from materials having suitable mechanical properties (such as puncture resistance, pin hole resistance, tensile strength), chemical properties (such as forming a suitable bond to the tube 16), and biocompatibility. In one embodiment, the walls of the inflatable cuff 12 are made of a polyurethane having suitable mechanical and chemical properties. An example of a suitable polyurethane is Dow Pellethane® 2363-80A. In another embodiment, the walls of the inflatable cuff 12 are made of a suitable polyvinyl chloride (PVC). Other suitable materials include polypropylene, polyethylene teraphthalate (PET), low-density polyethylene (LDPE), silicone, neoprene, or polyisoprene.

The water-swellable layer 14 is configured to be disposed on the outer, tissue-contacting surface of the cuff 12. FIG. 2 shows the exemplary cuffed endotracheal tube 10 inserted into a patient's trachea. As depicted, the water-swellable layer 14 may directly contact mucosal tissue that is involved in producing secretions that may travel into the lungs. The cuff 12 is inflated to form a seal against the tracheal walls 28 such that the water-swellable layer 14 is in contact with the mucosal tissue. Thus, mucosal secretions 30 are prevented from entering the lungs by the water-swellable layer.

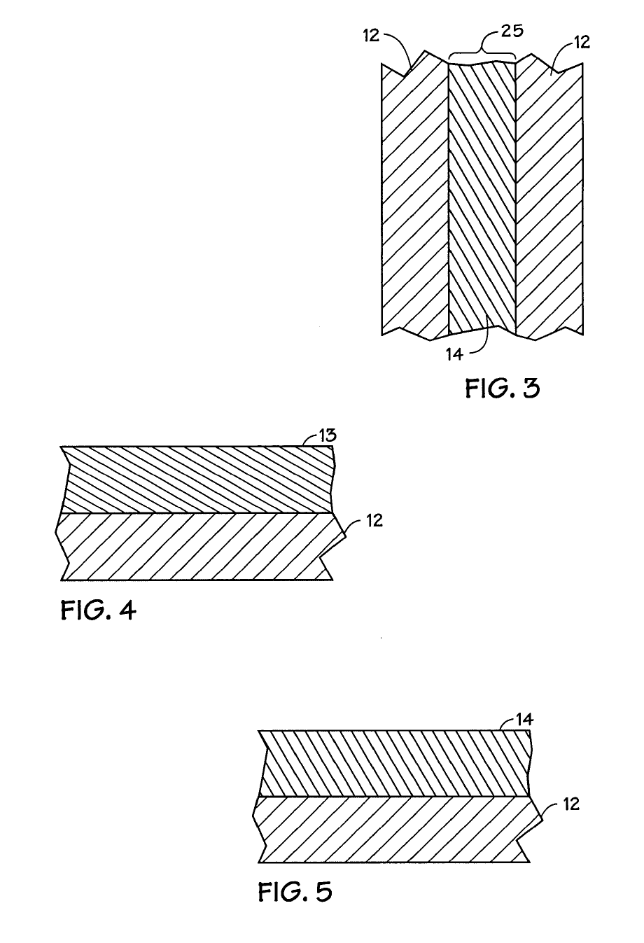

As shown in FIG. 3, the water-swellable layer 14 may be disposed on the cuff 12 such that as the cuff 12 folds in on itself to form wrinkles 25, the water-swellable layer 14 creates a vertical seal down the wrinkles 25. In such an embodiment, the hydrated water-swellable layer 14 may be a few microns to several millimeters in thickness in order to fill the fold of the cuff, depending on how the cuff 12 folds. In some embodiments, the water-swellable layer 14 is inserted into the trachea in the non-hydrated state and is swelled by the secretions and moisture in the trachea. However, there may be applications where waiting for natural body fluids to activate the non-hydrated cuff is contraindicated. In those applications, saline or other suitable source of moisture can be provided (e.g., via injection, dipping, or spraying) to the cuff 12, either immediately prior to insertion or immediately after insertion into the patient. In this way, the time required for full expansion of the water-swellable layer 14 may be reduced.

The water-swellable layer 14 may include any suitable water-swellable composition, such as those detailed in International Patent Application WO200623486 by Hadba el al., which is hereby incorporated by reference in its entirety herein. For example, the water swellable layer 14 may include hydrogels, polymers, or copolymer mixtures. In addition to being characterized by its composition, the water-swellable layer 14 may also be characterized by, for example, its level of adherence to the cuff 12. In certain embodiments, the water-swellable layer 14 may be loosely adhered or not adhered to the surface of the cuff 12. In such embodiments, the water-swellable layer 14 may be able to be squeezed out of areas where the cuff 12 seals tightly against the tracheal walls 28. Such an embodiment may be advadageous, as the squeezing may tend to result in a thicker build-up of the water-swellable layer 14 in areas in which the cuff 12 is not tightly sealed against the tracheal walls 28. In other embodiments, a non-adhered water-swellable layer 14 may be sufficiently cross-linked to reduce its flowability so that it is not squeezed out of folds or tissue contact areas by pressures that are typical of cuff inflation pressures. Such an embodiment may be advantageous in preventing the water-swellable layer 14 from dissolving away from the cuff 12, as the cross-linking may increase the robustness of the water-swellable layer 14.

In other embodiments, the water-swellable layer 14 may be adhered to the cuff 12. For example, the water-swellable layer 14 may form an interpenetrating network with the cuff 12. Such an interpenetrating network may be achieved by polymerizing the water-swellable layer 14 directly onto the cuff from a suitable monomer/oligomer solution, discussed in more detail below. While others have discussed chemical adhesion of a hydrogel to a cuff material through chemical bonding or simple diffusion of the gel into the cuff material, none of these previous cuffs have provided a cuff 12 with a hydrogel interpenetrating network. While an already polymerized hydrogel is capable of only limited diffusion into a cuff material, the relatively smaller monomers or oligomers as provided herein may readily diffuse into the cuff material. After their diffusion, a polymerization process may be initiated, which may form an interpenetrating polymer network within the cuff material. As shown in FIG. 4, a cuff 12 with an interpenetrating water-swellable layer 14 may form an interface layer 13 that includes a polymerized network that has penetrated the cuff material. The level of diffusion of the monomer or oligomers into the cuff material may determine the level of adhesion of the hydrogel to the cuff.

In other embodiments, the water-swellable layer,14 may be characterized by its swellability. For example, certain hydrogel compositions may be able to swell up to 100,000% of their non-hydrated size when fully hydrated. It is contemplated that the water-swellable compositions provided herein may swell up to 100%-100,000% when fully hydrated. In specific embodiments, the water-swellable layer 14 may swell up to 1000% of non-hydrated size. It should be understood that swellability of a hydrogel may be related to its cuff adherence. For example, a loosely adhered hydrogel may be able to swell to a greater extent than either a tightly adhered hydrogel or a hydrogel that has formed an interpenetrating network with the cuff 12.

In a specific embodiment, the water-swellable layer 14 may be a copolymer that includes repeating prepolymer units, e.g. one or more monomers, such as 3-sulfopropyl acrylate potassium salt (“KSPA”), sodium acrylate (“NaA”), N-(tris(hydroxy1methyl)methyl)acrylamide (“tris acryl”), 2-acrylamido-2-methyl-1-propane sulfonic acid (AMPS), or any combination thereof. The copolymer may include a first monomer and a second monomer that is different from the first monomer. The first monomer may be from 5 to 95% of the total monomer used to form the copolymer and the second monomer can be from 95 to 5% of the total monomer used to form the copolymer. In other embodiments, the second monomer can be from 75 to 25% of the total monomer used to form the copolymer. The water-swellable composition may also be formed from homopolymers that may include KSPA, NaA, trisacryl and AMPS. Other suitable monomers that may be incorporated into the water-swellable layer 14 may include 3-sulfopropyl methacrylate sodium salt (KSPMA), N-vinyl pyrrolidorie (NVP), allyl alcohol, allylamine, polyethylene glycol acrylate, polyethylene glycol methacrylate, vinyl functional phospholipids, and single or multiple vinyl functional conducting monomers (e.g. pyrrole), or any combination thereof. In certain embodiments, hydrophilicity modifying monomers or cationic monomers may be incorporated into the water-swellable layer 14 to control the swelling kinetics and/or the amphipathic character of the water-swellable layer 14. Such modifying monomers may include vinyl pyridine, methylmethacrylate, acrylated silicones, acrylated polypropylene glycol, acrylated poloxamers, butylacrylate, cyclohexylacrylate, styrene, styrene sulphonic acid, etc.

The water-swellable layer 14 may be cross-linked. A suitable cross-linker, if present, may be, for example, a low molecular weight di- or polyvinylic cross-linking agent such as ethylenglycol diacrylate or dimethacrylate, di-, tri- or tetraethylen-glycol diacrylate or dimethacrylate, allyl (meth)acrylate, a C2-C8-alkylene diacrylate or dimethacrylate, divinyl ether, divinyl sulfone, di- and trivinylbenzene, trimethylolpropane triacrylate or trimethacrylate, pentaerythritol tetraacrylate or tetramethacrylate, bisphenol A diacrylate or dimethacrylate, methylene bisacrylamide or -bismethacrylamide, ethylene bisacrylamide or ethylene bismethacrylamide, triallyl phthalate, diallyl phthalate, or N,N′methylenebisacrylamide (“MBAA”). When used, a cross-linking agent may be used in amounts from 0.1 to 20 percent by weight of the copolymer, and, in specific embodiments, may be used in amounts from 0.1 to 10 percent by weight of the copolymer.

The copolymer may be formed using any suitable technique with respect to the starting materials chosen. In certain embodiments, the copolymer may be prepared with the use of polymerization initiator. Suitable polymerization initiators are typically those that are initiating a radical polymerization of ethylenically unsaturated compounds. The radical polymerization may be induced thermally or by radiation (e.g., UV, visible, IR, y, E-beam and the like). In some embodiments, UV or visible light is used to induce polymerization. Redox initiation may also be used. Suitable thermal polymerization initiators are known to the skilled artisan and include for example peroxides, hydroperoxides, azobis(alkyl- or cycloalkylnitriles), persulfates, percarbonates or mixtures thereof. Examples are benzoylperoxide, tert-butyl peroxide, tert-butylperoxybenzoate, di-tert-butyl-diperoxyphthalate, tert-butyl hydroperoxide, 2,2′azobisisobutyronitrile, 1,1′-azobis(cyclohexanecarbonitrile), 4,4′-azobis(4-cyanovaleric acid) and the like. Initiators for the radiation-induced polymerization, so-called photoinitiators, fall into two groups based on the photochemical processes that lead to the production of radicals. These two groups are a-cleavage photoinitiators and hydrogen abstraction photoinitiators. Examples of a-cleavage initiators include benzoin ethers, hydroxy alkyl phenyl ketones, dialkoxy acetophenones, methyl thiophenyl morpholino ketones, phosphine oxide derivatives, morpholino phenyl amino ketones and benzoyl cyclonexanol. Examples of H-abstraction initiators include benzophenones, thioxanthones, benzyls, camphorquinones and ketocoumarins.

Water soluble photoinitiators may also be used in formation of the water-swellable layer 14. These are typically prepared by introducing water solubilizing groups onto the backbone of the initiator such that they do not significantly alter the activity of the initiator. These groups include quarternary ammonium salts, sulfonate groups, thiosulfate groups, carboxylic acid groups or hydrophilic chains. Some useful water soluble initiators are based on benzophenones, thioxanthones, benzyls, hydroxyl alkyl ketones, benzoyl methyl thiosulfate and phenyl trimethyl benzoyl phosphinates. Useful photoinitiators include for example benzophenones substituted with an ionic moiety, a hydrophilic moiety or both such as 4-trimethylaminomethyl benzophenone hydrochloride or benzophenone sodium 4-methanesulfonate; benzoin C1-C4 alkyl ether such as benzoin methyl ether; thioxanthones substituted with an ionic moiety, a hydrophilic moiety or both such as 3-(2-hydroxy-3-trimethylaminopropoxy)thioxanthone hydrochloride, 3-(3-trimethylaminopropoxy)thioxanthone hydrochloride, thioxanthone 3-(2-ethoxysulfonic acid) sodium salt or thioxanthone 3-(3-propoxysulfonic acid)sodium salt; or phenyl ketones such as 1-hydroxycyclohexylphenyl ketone, (2-hydroxy-2-propyl)(4-diethylene glycol phenyl)ketone, (2-hydroxy-2-propyl)(phenyl-4-butanecarboxylate)ketone; or commercial products such as those available under the tradenames DarocureB® or Irgacurem®. Using such initiators, copolymers may be polymerized in situ by long wavelength ultraviolet light or by light of about 5 14 nm, for example. The polymerization initiator can be present in an amount of, for example, 0.05 to about 5% by weight, based on the entire amount of monomer used. Another suitable photoinitiator is 2-hydroxy-1-(4-(2-hydroxyethoxy)-2-methyl-1-propanone (“HEMP”) available from Ciba Specialty Chemicals under the tradename IRGACURE®2959. In such embodiments, an aqueous solution containing the monomers (and optionally a cross-linking agent) and the photoinitiator may be prepared. The solution is then exposed to a suitable radiation source, such as a UV lamp, to effectuate polymerization.

In certain embodiments, the water-swellable layer 14 may be a porous hydrogel. Such hydrogels may be prepared by a solution polymerization technique, which entails polymerizing monomers in a suitable solvent. The nature of a synthesized hydrogel, whether a compact gel or a loose polymer network, depends on the type of monomer, the amount of diluent in the monomer mixture, and the amount of cross-linking agent. Porous hydrogels can be made by preparing hydrogels (usually from polymerizable monomers) in the presence of dispersed water-soluble porosigens, which can be removed to leave behind pores of a certain size. Examples of suitable porosigens are micronized sucrose, lactose, and dextrin, sodium chloride, and poly(ethylene oxides) (PEGs).

Optionally, therapeutically beneficial compounds may be incorporated into the water-swellable layer 14. The biologically-active agent may be soluble in the polymer solution to form a homogeneous mixture, or insoluble in the polymer solution to form a suspension or dispersion. Over time, the biologically-active agent may be released from the cuff 12 into the adjacent tissue fluids, for example at a controlled rate. The release of the biologically-active agent from the present composition may be varied, for example, by the solubility of the biologically-active agent in an aqueous medium, the distribution of the agent within the composition, ion exchange, pH of the medium, the size, shape, porosity, solubility and biodegradability of the article or coating, and the like. The term “therapeutically beneficial compound” encompasses therapeutic agents, such as drugs, and also genetic materials and biological materials.

A variety of therapeutically beneficial compounds may be included, such as those detailed in International Patent Application WO200623486 by Hadba et al. For example, the therapeutically beneficial compound may include proteins (including enzymes, growth factors, hormones and antibodies), peptides, organic synthetic molecules, inorganic-compounds, natural extracts, nucleic acids (including genes, telomerase inhibitor genes, antisense nucleotides, ribozymes and triplex forming agents), lipids and steroids, carbohydrates (including heparin), glycoproteins, polymeric drugs, e.g. polysalicilic acid, prodrugs, and combinations thereof. The therapeutically beneficial compound may have a variety of biological activities, such as vasoactive agents, neuroactive agents, hormones, anticoagulants, immunomodulating agents, cytotoxic agents, antibiotics, antivirals, or may have specific binding properties such as antisense nucleic acids, antigens, antibodies, antibody fragments or a receptor. Proteins including antibodies or antigens can also be delivered. Proteins are defined as consisting of 100 amino acid residues or more; peptides are less than 100 amino acid residues. Unless otherwise stated, the term protein refers to both proteins and peptides. Examples include insulin and other hormones.

The cuff 12 may be manufactured by any suitable method, including extrusion, co-extrusion, spraying, dipping, coating, or deposition. For example, cuff 12 as provided herein may be manufactured by an extrusion process. In such a process, the cuff 12 may be made by using extruded or pre-extruded tubing and applying heat and pressure appropriately within a molding cavity to achieve the desired shape (blow molding). Cuff 12 may also be formed by extrusion blow molding, wherein an extruder fed polymer pellets melts the polymer and feeds the molten polymer through a die to form a tube shape. This still molten polymer is then captured in a mold and air pressure is applied to expand the tube out to the walls of the mold, thus achieving the desired shape. In the extrusion blow molding process, a core or mandrel of the extruder has apertures to admit a gas such as pressurized air or an inert gas like nitrogen, into the medical device in the neighborhood of the cuff. After a length of medical device has been extruded, a mold clamps the medical device around the mandrel. As gas is admitted to the cuff area through the mandrel, the cuff expands against the mold. In the alternative, the cuff wall may be expanded in a second discrete expansion process following an extrusion or molding process, such as with a shuttle blow molding process. After initial extrusion, the extruded cuff 12 will have a generally tubular shape with a substantially uniform wall thickness. This tubular shape may then be blown into the tapered shape. This process results in the area of the cuff with larger diameters having thinner walls because the same amount of material is stretched over a larger area. A programmable parison allows the wall thickness being extruded to be controlled as a function of length.

The water-swellable layer 14 may be applied to the cuff 12 by any suitable method, such as extrusion, co-extrusion, spraying, dipping, coating, or deposition. Further, the water-swellable layer 14 may be polymerized in place on the cuff 12, or may be applied to the cuff 12 as a polymerized sheet or layer. In embodiments in which the water-swellable layer 14 is adhered to the cuff 12, the cuff 12 may be soaked in an appropriate monomer or oligomer solution that may be polymerized in such a manner as to adhere to the cuff surface. Alternatively, the water-swellable layer 14 may be extruded over the cuff 12.

In one embodiment, a cuff 12 with an interpenetrating water-swellable layer 14 may be formed by soaking the cuff in a monomer or oligomer solution that has been swelled in an appropriate solvent, such as a solvent that is miscible with water, for a period of time prior to polymerization. The solvent may also include appropriate cross-linkers and initiators. The swollen solution may be treated with heat or radiation to initiate the polymerization of the monomers or oligomers into an interpenetrating network of a hydrogel in the cuff material. The choice of solvent system, monomer concentration, surface treatment of the cuff material, modifications made in bulk (e.g. adding monomer without solvent) swelling time, and polymerization conditions may determine the depth of penetration of the interpenetrating network and the coating thickness.

In a specific embodiment, a cuff 12 may be dipped in a 20% monomer solution of 25% KSPA and 75% NaA. The monomers may be mixed with N,N′-Methylenebisaacrylamide as a cross-linker and 2-hydroxy-4′-(2-hydroxyethoxy)-2-methylpropiophenone as a UV initiator. The cross-linker may be generally added to the monomer solution at a concentration of less than 2% of the monomer, and the initiator may be added to the solution at a concentration of 0.1% of the monomer. Alternately, a heat initiator such as 2,2′-Azobisisobutyronitrile may be used in place of the UV initiator. The cuff 12 may be dipped in the monomer solution either after before or after any further cuff processing (such as blow molding). After dipping, the cuff may be exposed to UV light for 10 seconds to 5 minutes. During the UV exposure, the cross-linker converts the monomer into a hydrogel polymer that may swell up to 40,000% of its non-hydrated size. In specific embodiments, the hydrogel polymer may swell 1000% to 40,000% of its non-hydrated size.

FIGS. 5-7 illustrate alternate configurations of a water-swellable layer 14 on a cuff 12. FIG. 5 illustrates a configuration in which a water-swellable layer 14 is applied directly to a polyvinyl chloride cuff 12. The water-swellable layer 14 may be applied to the cuff 12 as a dehydrated layer that may swell upon exposure to water. Additionally, the cuff 12 including the water-swellable layer 14 may be shipped in the dehydrated state so that a healthcare worker may hydrate the water-swellable layer 14 close to the time of cuff insertion. The water-swellable layer 14 may also be applied by the healthcare worker as a liquid or gel that is not adhered or is loosely adhered to the cuff 12.

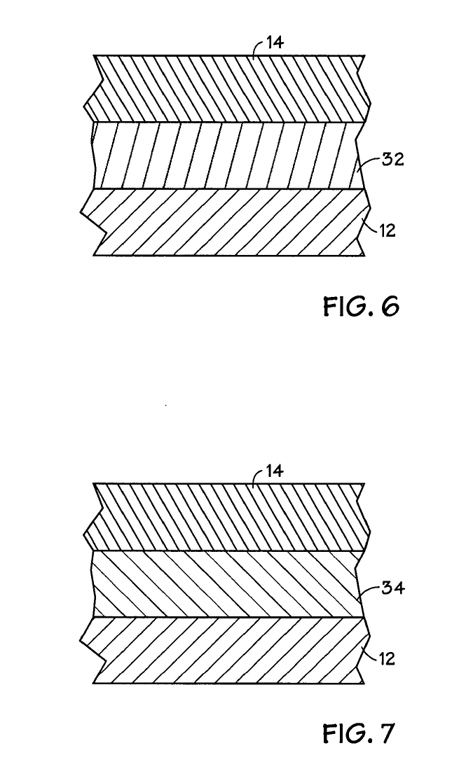

The cuff 12 may also be surface-treated prior to applying the water-swellable layer 14. The surface treatment may include plasma treatment, corona discharge, ion implantation, ion bombardment, or treatment with chemical coupling agents (e.g. silane coupling agents, Volan), surfactants, or primers. The surface treatment, which may alter the chemistry or material properties of the surface of the cuff material, may enhance the adhesion of the water-swellable layer 14 to the cuff 12. FIG. 6 illustrates an exemplary cuff 12 that includes a surface tie layer 32. Suitable tie layers include polyvinyl pyrrolidone. The tie layer may be coextruded with the cuff 12. FIG. 7 illustrates a cuff with a hydromer surface coating 34. A water-swellable layer 14 may be applied to the hydromer coating 34. The hydromer coating 34 may provide a more hydrophilic surface on the polyvinyl chloride cuff 12. Accordingly, the water-swellable layer 14 may be applied more easily to the relatively more hydrophilic hydromer coating 34.

The tracheal cuffs of the present techniques may be incorporated into systems that facilitate positive pressure ventilation of a patient, such as a ventilator. Such systems may typically include connective tubing, a gas source, a monitor, and/or a controller. The controller may be a digital controller, a computer, an electromechanical programmable controller, or any other control system.

Typically, low pressure endotracheal cuffs are inflated within a patient's trachea such that the intra cuff pressure is approximately 20-25 cm H₂O. Endotracheal cuffs utilizing inflation pressures significantly greater 50 cm H₂O may be referred to as high-pressure cuffs, while cuffs that are able to effectively seal the trachea at pressures less than 30 cm H₂O may be considered low-pressure cuffs. In certain embodiments, intra cuff inflation pressures of 10-30 cm H₂O may be used with the cuffs of the present techniques.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1-13. (canceled)

14. A medical device comprising:
an inflatable balloon cuff comprising a distal opening and a proximal opening, wherein the distal opening and the proximal opening are suitably sized to accommodate a conduit; and
a composition disposed on the balloon cuff, the composition comprising a copolymer that includes a first monomer, and a second monomer that is different from the first monomer, wherein the first monomer comprises 3-sulfopropyl acrylate potassium salt, sodium acrylate, N-(tris(hydroxyl methyl)methyl)acrylamide, or 2-acrylamido-2-methyl-1-propane sulfonic acid.

15. The medical device, as set forth in claim 14, wherein the first monomer is 3-sulfopropyl acrylate potassium salt and the second monomer is sodium acrylate.

16. The medical device, as set forth in claim 14, wherein the first monomer is 3-sulfopropyl acrylate potassium salt and the second monomer is N-(tris(hydroxyl methyl)methyl)acrylamide.

17. The medical device, as set forth in claim 14, wherein the first monomer is 3-sulfopropyl acrylate potassium salt and the second monomer is 2-acrylamido-2-methyl-1-propane sulfonic acid.

18. The medical device, as set forth in claim 14, wherein the first monomer is sodium acrylate and the second monomer is N-(tris(hydroxyl methyl)methyl) acrylamide.

19. The medical device, as set forth in claim 14, wherein the first monomer is sodium acrylate and the second monomer is 2-acrylamido-2-methyl-1-propane sulfonic acid.

20. The medical device, as set forth in claim 14, wherein the first monomer is N-(tris(hydroxyl methyl)methyl)acrylamide and the second monomer is 2-acrylamido-2-methyl-1-propane sulfonic acid.

21. The medical device, as set forth in claim 14, comprising a hydrophilicity modifying monomer.

22. The medical device, as set forth in claim 21, wherein the hydrophilicity modifying monomer is selected from the group consisting of methylmethacrylate, butylacrylate, cyclohexylacrylate, styrene, styrene sulphonic acid, or any combination thereof

23-27. (canceled)

28. A method of manufacturing a medical device, comprising:
providing an inflatable balloon cuff comprising a distal opening and a proximal opening, wherein the distal opening and the proximal opening are suitably sized to accommodate a conduit; and
disposing a composition on the balloon cuff, the composition comprising a copolymer that includes a first monomer, and a second monomer that is different from the first monomer, wherein the first monomer comprises 3-sulfopropyl acrylate potassium salt, sodium acrylate, N-(tris(hydroxyl methyl)methyl)acrylamide, or 2-acrylamido-2-methyl-1-propane sulfonic acid.

29. The method as set forth in claim 28, wherein disposing the composition on the balloon cuff comprises dipping the balloon cuff in a solution comprising the composition.

30. The method as set forth in claim 28, wherein disposing the composition on the balloon cuff comprises dipping the balloon cuff in a solution comprising the first monomer and the second monomer; and
polymerizing the first monomer and the second monomer on the cuff

31. The method as set forth in claim 28, wherein disposing the composition on the balloon cuff comprises spraying the balloon cuff with a solution comprising the composition.

32. The method as set forth in claim 28, wherein disposing the composition on the balloon cuff comprises spraying the balloon cuff with a solution comprising the first monomer and the second monomer; and
polymerizing the first monomer and the second monomer on the cuff

33. A medical device comprising:
an inflatable balloon cuff comprising a distal opening and a proximal opening, wherein the distal opening and the proximal opening are suitably sized to accommodate a conduit; and
a water-swellable interpenetrating network disposed on the balloon cuff

34. The medical device, as set forth in claim 33, wherein the balloon cuff comprises polyethylene teraphthalate (PETP), low-density polyethylene (LDPE), polyvinyl chloride (PVC), silicone, neoprene, polyisoprene, or polyurethane (PU).

35. The medical device, as set forth in claim 33, comprising an endotracheal tube associated with the balloon cuff, wherein the endotracheal tube passes through the proximal opening and the distal opening of the balloon cuff

36. The medical device, as set forth in claim 35, wherein the endotracheal tube is operatively connected to a ventilator.

37. The medical device, as set forth in claim 33, wherein the monomer or oligomer comprises at least one of 3-sulfopropyl acrylate potassium salt, N-(tris(hydroxyl methyl)methyl)acrylamide, 2-acrylamido-2-methyl-1-propane sulfonic acid, sodium acrylate, 3-sulfopropyl methacrylate sodium salt, N-vinyl pyrrolidone, allyl alcohol, allylamine, polyethylene glycol acrylate, polyethylene glycol methacrylate, vinyl functional phospholipids, single or multiple vinyl functional conducting monomers, or any combination thereof

38. The medical device, as set forth in claim 33, comprising a therapeutically beneficial compound.

39. The medical device, as set forth in claim 33, comprising a hydrophilicity modifying monomer.

40. The medical device, as set forth in claim 39, wherein the hydrophilicity modifying monomer comprises at least one of methylmethacrylate, butylacrylate, cyclohexylacrylate, styrene, styrene sulphonic acid, or any combination thereof.

41. The medical device, as set forth in claim 33, wherein the balloon cuff comprises a polyvinyl pyrrolidone tie layer disposed on the surface of the balloon cuff

42. The medical device, as set forth in claim 33, wherein the balloon cuff comprises a hydromer layer disposed on the surface of the balloon cuff

43. The medical device, as set forth in claim 33, wherein the water-swellable coating is adapted to swell to at least 40,000% of its dehydrated size when fully hydrated.

44. A method of manufacturing a medical device, comprising:
providing an inflatable balloon cuff comprising a distal opening and a proximal opening, wherein the distal opening and the proximal opening are suitably sized to accommodate a conduit;
contacting the balloon cuff with a swelled prepolymer solution comprising at least one monomer or at least one oligomer, at least one cross-linker, and at least one initiator, wherein the at least one monomer or the at least one oligomer are adapted to form a hydrogel when polymerized;
polymerizing the swelled prepolymer solution to form an interpenetrating hydrogel network on the balloon cuff

45. The method as set forth in claim 44, wherein contacting the balloon cuff with a swelled prepolymer solution comprises dipping or soaking the balloon cuff in the solution for a predetermined amount of time.

46. The method as set forth in claim 45, wherein the predetermined amount of time is 10 seconds to 5 minutes.
